(19) United States
(12) Patent Application Publication
(10) Pub. No.: US 2026/0248561 A1
(43) Pub. Date: Aug. 27, 2026

(54) MACHINE LEARNING FOR OBJECT TRACKING

(71) Applicant: Cornell University, Ithaca, NY (US)

(72) Inventors: Bobak Mosadegh, New York, NY (US); Mohsen Annabestani, Ithaca, NY (US)

(73) Assignee: Cornell University, Ithaca, NY (US)

(21) Appl. No.: 19/162,724

(22) PCT Filed: Mar. 5, 2024
(86) PCT No.: PCT/US2024/018511

§ 371 Date: Sep. 5, 2025

Related U.S. Application Data
(60) Provisional application No. 63/488,641, filed on Mar. 6, 2023.

(51) Int. Cl. A61B 34/20; A61B 6/00; A61B 6/46; G06T 7/73

(52) U.S. Cl. CPC A61B 34/20; A61B 6/461; A61B 6/547; G06T 7/73

(57) ABSTRACT

The present disclosure provides systems and methods capable of determining a roll angle of a medical instrument in real time based on one or more medical images that include the medical instrument. In different versions, the images may be, for example, fluoroscopic, ultrasound, and/or video. The roll angle may be determined using one or more machine learning models. Other degrees of freedom may also be determined. The roll angle (and potentially other degrees of freedom) may be used in visualizations that guide a user of the medical instrument. The visualization may employ various displays and/or headsets, and may be used with an augmented reality (AR) system, a mixed reality (MR) system, a virtual reality (VR) system, an extended reality (XR) system, and/or a spatial computing system.

### CROSS-REFERENCE TO RELATED APPLICATIONS

This application claims the benefit of and priority to U.S. Provisional Application No. 63/488,641 filed Mar. 6, 2023, the contents of which are incorporated by reference in their entirety for any and all purposes.

### TECHNICAL FIELD

Embodiments of the present technology relate to systems and methods for characterization and visualization of a medical instrument and determination of relative positioning or orientation of the instrument, such as determining or predicting roll angle of a medical instrument, employing machine learning techniques, in real-time or near real time while the instrument is being handled or otherwise used.

### BACKGROUND

Objects may be capable of exhibiting motions with multiple degrees of freedom, resulting from up-down, left-right, forward-back, roll, pitch, and yaw. The ability to move in these six distinct ways may be crucial for aircraft, robotic systems, and augmented/virtual reality (AR/VR) systems.

### SUMMARY

In one aspect, various embodiments relate to a medical imaging system comprising one or more processors, the system configured to: receive, in real time, one or more medical images from an imaging device, the one or more medical images including one or more depictions of a medical instrument; determine, based at least in part on at least one of the received medical images, a roll angle of the medical instrument, the roll angle being indicative of a position of a reference feature of the medical instrument relative to a target; and generate a visualization based at least in part on the determined roll angle for presentation via a display device.

In various embodiments, the system is configured to determine the roll angle of the medical instrument based at least in part on a machine learning model.

In various embodiments, the machine learning model was trained based on groundtruth roll angle measurements. The groundtruth roll angle measurements may have been acquired from one or more of: (i) pairs of images taken at two different angles, (ii), electromagnetic sensors, (iii) impedance sensors, (iv) ultrasound crystals, and/or (v) fiber optics.

In various embodiments, the machine learning model was trained using a set of training data, each data point associated with a label indicative of roll angle for the medical instrument depicted in the image.

In various embodiments, the machine learning model comprises one or more deep neural networks.

In various embodiments, the machine learning model was trained to determine roll angle based on single images.

In various embodiments, the roll angle is a first degree of freedom (DOF), the medical imaging system further configured to determine one or more additional degrees of freedom (DOFs).

In various embodiments, the roll angle is a first degree of freedom (DOF), the medical imaging system further configured to determine five additional degrees of freedom (DOFs).

In various embodiments, the medical instrument is a minimally invasive tool, a catheter, and/or imaging hardware. In various embodiments, the imaging hardware may be or may comprise one or more endoscopic devices.

In various embodiments, the one or more medical images are based at least in part on fluoroscopy.

In various embodiments, the one or more medical images are based at least in part on ultrasound.

In various embodiments, the one or more medical images are based at least in part on digital images captured using image sensors.

In various embodiments, the visualization is for an augmented reality (AR) system, a mixed reality (MR) system, a virtual reality (VR) system, an extended reality (XR) system, or a spatial computing system that interfaces with the display screen, a second display screen, and/or a headset.

In another aspect, various embodiments relate to a method comprising: applying, in real time, a machine learning model to one or more medical images to determine a roll angle of a medical instrument depicted in the medical images; wherein the roll angle of the medical instrument is indicative of a position of a reference feature of the medical instrument relative to a target; and wherein the one or more medical images are based on one or more of fluoroscopy, ultrasound, or digital imagery.

In various embodiments, the method may comprise generating a visualization based at least in part on the determined roll angle for presentation via a display device of an augmented reality (AR) system, a mixed reality (MR) system, a virtual reality (VR) system, an extended reality (XR) system, and/or a spatial computing system.

In various embodiments, the method comprises training the machine learning model.

In yet another aspect, various embodiments relate to a method comprising training a machine learning model to determine roll angle of a medical instrument based on single medical images.

In various embodiments, the method comprises using a trained machine learning model to determine roll angle of a medical instrument.

In various embodiments, the roll angle is determined in real time or near real time during a medical procedure.

### BRIEF DESCRIPTION OF THE DRAWINGS

FIG. 1 is an example system comprising components capable of implementing various illustrative embodiments of the disclosure.

FIG. 2 depicts example processes for implementing various embodiments of the disclosed approach, according to various illustrative embodiments of the disclosure.

FIG. 3 depicts, at (a), an Intracardiac Echocardiography (ICE) catheter sensor head, at (b), fluoroscopy imaging of the ICE catheter sensor head, and at (c), a three-dimensional (3D) representation of Antero Posterior (AP) and Left Anterior Oblique at 90 degrees (LAO90) planes, according to various illustrative embodiments of the disclosure.

FIG. 4 depicts an example a low fidelity (LF) and high fidelity (HF) machine learning model block diagram, according to various illustrative embodiments of the disclosure.

FIG. 5 depicts an example procedure for extracting features for three samples (the first, mth and nth frames) at point P1 of both AP and LAO90 planes, according to various illustrative embodiments of the disclosure.

FIGS. 6A and 6B depict Horizontal and Vertical Displacement of P1 and P2 in AP (FIG. 5A) and LAO90 (FIG. 5B) planes, according to various illustrative embodiments of the disclosure.

FIG. 7 depicts, at (a), Euclidean distance (D) of the black width (between points q1 and q2) of the ICE catheter tip on the AP plane, and at (b), distance “D” and the extracted roll angle of the ICE catheter as the ground truth data for the machine learning (ML) model, according to various illustrative embodiments of the disclosure.

FIG. 8 depicts actual and prediction for roll angle (in degrees) for a blind test dataset using an example LF/HF ML model, according to various illustrative embodiments of the disclosure.

FIG. 9 depicts error criteria for three sample groups: Training and Validation samples, Blind test samples, and all samples (Training, Validation, and Blind test). At (a), FIG. 8 depicts Normalized Mean Square Error (NMSE), at (b), Mean Absolute Error (MAE), and Standard Deviation of Absolute Error (SDAE), at (c-e) Error histograms, and (f-h) Linear correlation and R-squared, according to various illustrative embodiments of the disclosure.

FIG. 10 provides a block diagram of an example vision algorithm, according to various illustrative embodiments of the disclosure.

FIG. 11 depicts an example Cubic 3D setup, with a 3D Designed version on the left, and a 3D Printed Version on the right, according to various illustrative embodiments of the disclosure.

FIGS. 12A and 12B depict the 3D positional displacement of four tracked points on the ICE catheter head during various experiments conducted along the defined heart boundary, according to various illustrative embodiments of the disclosure. These experiments aim to encompass the entire boundary of the heart, forming a comprehensive training dataset for example embodiments of the machine learning (ML) model.

FIG. 13 depicts the concatenation of all data presented in FIGS. 12A and 12B, according to various illustrative embodiments of the disclosure. Embodiments of the machine learning (ML) model may undergo training using such a concatenated dataset to eliminate biases related to the specific location of the heart boundary, resulting in improved generalization capabilities.

FIG. 14 depicts an example machine learning (ML) model designed for predicting the Z coordinate based on the input of X and Y coordinates, according to various illustrative embodiments of the disclosure. This model enables biplane 3D positional tracking, utilizing solely monoplane measurements.

FIG. 15 depicts the Actual and Predicted Z coordinates obtained through embodiments of the disclosed ML model, according to various illustrative embodiments of the disclosure. The left side displays results from the blind test dataset, while the right side presents a comprehensive view, including training, validation, and test datasets.

FIG. 16 depicts error criteria for three sample groups: Training and Validation samples, Blind test samples, and all samples (Training, Validation, and Blind test), according to various illustrative embodiments of the disclosure. (a) Mean Absolute Error (MAE), and Standard Deviation of Absolute Error (SDAE), (b) Normalized Mean Square Error (NMSE), (c-e) Error histograms.

FIG. 17 depicts a block diagram of a representative server system and client computer system usable to implement various embodiments of the present disclosure.

### DETAILED DESCRIPTION

It is to be appreciated that certain aspects, modes, embodiments, variations and features of the present methods are described below in various levels of detail in order to provide a substantial understanding of the present technology. The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

The disclosed approach enables characterization and visualization of various instruments. The characterization and visualization may relate to a relative positioning or orientation of the instrument. The instrument may have an asymmetry such that information about a roll angle of the instrument may have significance. Various embodiments of the disclosed approach include determining (e.g., predicting, inferring, and/or calculating) roll angle of a medical instrument. Other degrees of freedom of the medical instrument may be determined as well. The determination of roll angle may employ one or more machine learning models. Roll angle may be determined in real-time or near real time while the medical instrument is being manipulated or otherwise handled (e.g., by a clinician as part of a medical procedure). The instrument may be captured by images in one or more imaging modalities (e.g., fluoroscopy, ultrasound, and/or digital imagery using a video camera), and the images may be used to determine roll angle, such as by employing one or more machine learning models. The roll angle, or a representation or derivation thereof, may be presented visually or otherwise. In various embodiments, a visual representation may be provided on a display screen and/or through an augmented reality (AR) system, a mixed reality (MR) system, and/or a virtual reality (VR) system.

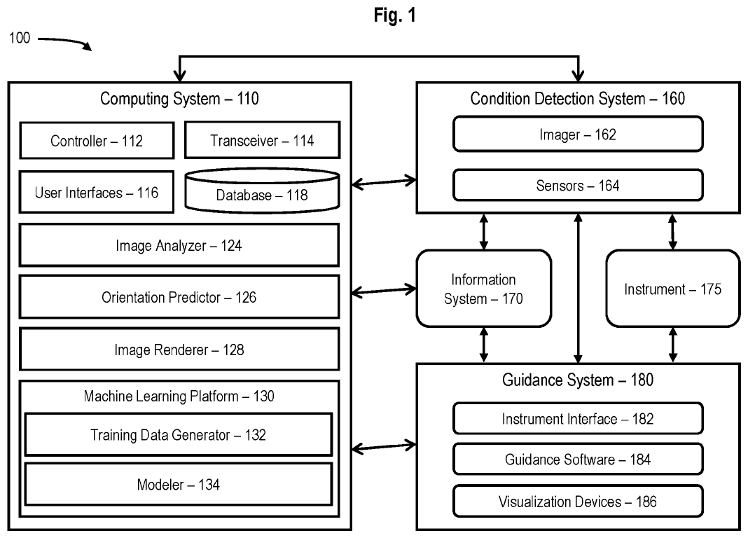

Referring to FIG. 1, in various embodiments, a system 100 may include a computing system 110 (which may be or may include one or more computing devices, co-located or remote to each other), a condition detection system 160 (capable of, e.g., obtaining data on a subject such as a human or non-human patient), an information system 170 (such as a management information system that may record and/or provide health information), instruments 175 (e.g., a medical instrument such as a catheter, a movable platform on which a patient may be situated, etc.), and a guidance system 180 (e.g., hardware and software that may be guide a clinician during use of an instrument). The computing system 110 (e.g., one or more computing devices) may be used to control and/or exchange signals and/or other data with condition detection system 160, information system 170, instrument 175, and/or guidance system 180, directly (e.g., through wireless and/or wired communication) or indirectly via another component of system 100 (e.g., via any combination of wireless and/or wired communication). The computing system 110 may include one or more processors and one or more volatile and/or non-volatile memories for storing computing code and data that are captured, acquired, recorded, and/or generated.

The computing system 110 may include a controller 112 that is configured to exchange control signals with condition detection system 160, information system 170, instrument 175, guidance system 180, and/or any components thereof, allowing the computing system 110 to be used to control, for example, capture of images, acquisition of signals by sensors, positioning or repositioning of subjects or devices, recording or obtaining other information, etc.

A transceiver 114 allows the computing system 110 to exchange readings, control commands, and/or other data or signals, wirelessly or via wires, directly or indirectly via networking protocols, with, for example, condition detection system 160, information system 170, instrument 175, and/or guidance system 180, or components thereof. One or more user interfaces 116 allow the computing device 110 to receive user inputs (e.g., via keyboard, touchscreen, microphone, camera, motion detection, biometric scan, etc.) and provide outputs (e.g., via display screens, audio speakers, light emitters, AR/VR/MR headsets etc.) with users. The computing device 110 may additionally include one or more databases 118 for storing, for example, data acquired from one or more systems or devices, signals acquired via one or more sensors, images, biomarker signatures, etc. In some implementations, database 118 (or portions thereof) may alternatively or additionally be part of another computing device that is co-located or remote (e.g., via “cloud computing”) and in communication with computing device 110, condition detection system 160, information system 170, instrument 175, and/or guidance system 180 or components thereof.

Condition detection system 160 may include one or more imagers 162, which may be or may include, for example, any system or device that is involved in, for example, capturing images prior to, during, or following a procedure. Imagers may employ any suitable imaging modality, such as fluoroscopy, ultrasound, or digital imagery from a camera. Imager 162 may include detectors for visible light and/or light in any frequencies of interest, such as (but not limited to) the spectrum from infrared to ultraviolet. Imagers may employ any suitable optical components (e.g., lenses, mirrors, filters, beam splitters, prisms, diffusers, diffraction gratings, etc.), digital components (e.g., charge-coupled devices (CCDs), as well as an area for placement of samples, computing components (e.g., one or more processors, such as digital signal processors) to process and/or pre-process images, etc. Such imagers may be incorporated into endoscopic devices. Imagers may be, or may employ, any devices, tools, and/or techniques that will provide the desired imaging data, such as an ultrasound transducer. The imager 162 may have the capability of receiving control signals from a computing device or system to, for example, initiate or cease image capture, and/or return images or other imaging data or status signals to the computing device or system (e.g., computing system 110). Sensors 164 may detect, for example, other aspects of the subject, instrument, and/or environment, such as position, motion, temperature, humidity, etc. In certain embodiments, sensors 164 may include devices for detecting electrical properties (e.g., devices for impedance tracking).

Guidance system 180 may include any components used to plan for and/or implement any procedures (e.g., a medical procedure using one or more instruments 175). Instrument interface 182, for example, may include any coupling of an instrument 175 with guidance system 180 that allows the guidance system 180 to, for example, communicate with (wirelessly or via wires) and/or control instrument 175. Instrument 175 and/or instrument interface 182 may include any combination of actuators, motors, robotic components, end effectors like grippers and manipulators, receiver and/or transmitter, etc. Guidance system 180 may also include or employ guidance software 184 that may, for example, receive inputs from users (e.g., selections regarding what information or visualization is presented, settings of instruments 175, etc.) and/or from other components of system 100 and provide or direct signals to users or and/or to other components of system 100, such as visualization devices 186. In various embodiments, visualization devices 186 may include any combination of display devices, headsets, and/or other devices that enable visual presentation of information.

In various implementations, components of system 100 may be rearranged or integrated in other configurations. For example, computing system 110 (or components thereof) may be integrated with one or more of the condition detection system 160, guidance system 180, instrument 175, and/or components thereof. The condition detection system 160, guidance system 180, and/or components thereof may be directed to an instrument 175 (e.g., a medical instrument or platform on which a patient is situated during a procedure). In various embodiments, the instrument 175 may be movable (e.g., using any combination of motors, magnets, etc.) to allow for positioning and repositioning (such as micro-adjustments for positioning of device or subjects). It is also noted that not all components of system 100 are required to implement the disclosed approach, and in various embodiments, only a subset of the components of system 100 may be employed. For example, in various embodiments, computing system 110 may obtain and process data that was obtained via an another system that is, or is not, in direct communication with the computing system 110.

An image analyzer 124 may retrieve or otherwise receive images (e.g., from or via condition detection system 160) and analyze or otherwise process the images to extract relevant information. Orientation predictor 126 may use raw or processed data (e.g., raw imaging data or imaging data obtained from or via image analyzer 124) to determine one or more degrees of freedom (DOFs) of the instrument 175. The orientation predictor 126 may be, or may comprise, a roll angle predictor that determines a roll angle (and/or another metric corresponding to other DOFs), such as by using machine learning models to calculate, predict, or infer roll angle of instrument 175. Image renderer 128 may render or otherwise generate images, elements of images, and/or other visualizations, which may be provided to, for example, guidance system 180 for presentation to a user via, for example, visualization devices 186.

Machine learning platform 130 may be configured to train and update machine learning models, as further discussed herein. Machine learning platform 130 may, for example, employ certain machine learning techniques and algorithms to train and update predictive models. Machine learning platform 130 may include a training data generator 132 which may, for example, generate or otherwise obtain training data, such as images or imaging data and/or labels that identify a characteristic of components of the images (e.g., labels identifying a roll angle of an instrument in training images). The modeler 134 may use training data to generate models that may be used for, for example, determining roll angle.

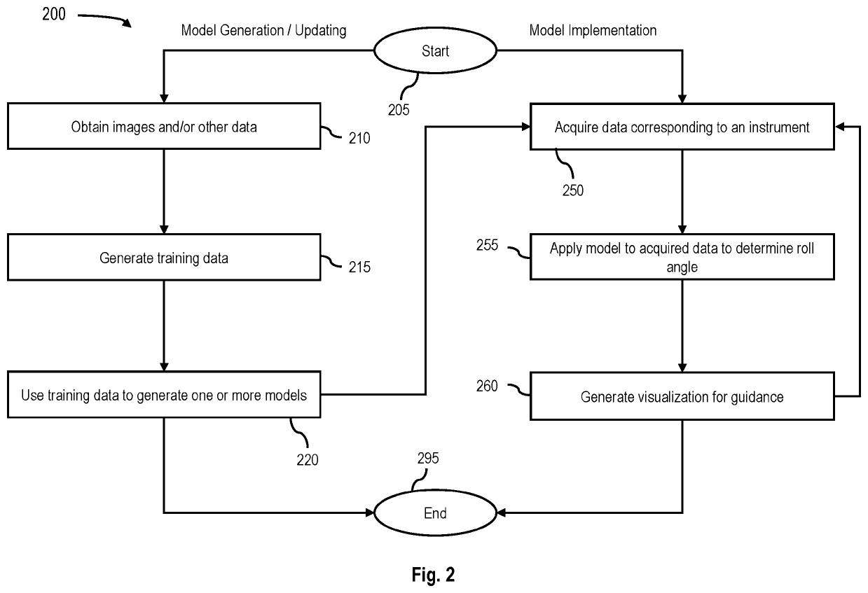

Referring to FIG. 2, an example process 200 is illustrated, according to various example embodiments. Various elements of process 200 may be implemented by or via system 100 or components thereof. On the left side is a model generation and/or updating subprocess, and on the right side is a model implementation subprocess. In various embodiments, a process 200 that includes blocks 250, 255, and 260 may be performed, without performing blocks 210, 215, and 220. In various other embodiments, a process 200 that includes blocks 210, 215, and 220 may be performed, without performing blocks 250, 255, and 260. In some embodiments, a process 200 that includes blocks 210, 215, 220, 250, 255, and 260 may be performed. Process 200 may begin at block 205 and either proceed to blocks 210, 215, 220, and 295 (e.g., if a model is to be trained or updated), or begin at block 205 and proceed to blocks 250, 255, 260, and 295 (e.g., if an available model is to be implemented).

In various embodiments, process 200 may begin at block 210 with obtaining images and/or other data to be used for training, updating, and/or otherwise generating a machine learning model. The data obtained at block 210 may, at block 215, be processed to generate training data suitable for generation of the model. For example, single images or pairs of images may be labeled or otherwise processed to obtain a suitable structure for the training data. At block 220, the training data may be used to generate one or more models. Process 200 may then end by proceeding to block 295, or continue to block 250 for model implementation.

In various embodiments, process 200 may begin at block 250 (or proceed to block 250 from block 220) with acquisition of data (e.g., imaging data) that includes or otherwise corresponds to a medical instrument. The data may include, for example, one or more images obtained during a medical procedure during which the medical instrument is used. At block 255, process 200 includes applying a machine learning model to the acquired data to determine roll angle. Other DOFs may also be determined. At block 260, process 200 includes generating a visualization to guide the user of the medical instrument. The visualization may be provided in real time (used interchangeably with near real time) while a subject is undergoing a procedure with the medical instrument. Process 200 may then end by proceeding to block 295. In some embodiments, blocks 210, 215, and 220 may be repeated to update the model based on, for example, different training data or varied model architecture.

Various embodiments of the disclosed approach may be illustrated through examples involving a catheter (e.g., one that may be employed in a catheterization procedure), used with an imaging modality such as intracardiac echocardiography (ICE), and a machine learning model with a multi-component architecture that employs deep neural networks trained on certain types of images. However, it should be understood that these examples are not intended to be limiting. Other embodiments may involve, alternatively or additionally, for example, other imaging modalities, other instruments, other model architectures, other model training techniques, other training data that may be generated differently, and/or other representations (visual and/or non-visual) of the orientation and/or position of instruments.

Catheterization is a procedure used to diagnose and treat various cardiovascular diseases. Intracardiac echocardiography (ICE) is an imaging modality that has gained popularity in these procedures due to its ability to provide high-resolution images of the heart and its surrounding structures in a minimally invasive manner. However, given its limited field of view, understanding its orientation within the heart is difficult simply from observing the acquired images. Therefore, ICE catheter tracking, which requires six degrees of freedom, would be useful to better guide interventionalists during a procedure. This work demonstrates a machine learning-based approach that has been trained to predict the roll angle of an ICE catheter using landmark scalar values extracted from bi-plane fluoroscopy images. The model consists of two fully connected deep neural networks that were trained on a dataset of bi-plane fluoroscopy images acquired from a 3D printed heart phantom. The results showed high accuracy in roll angle prediction, suggesting the ability to achieve 6 degrees of freedom tracking using bi-plane fluoroscopy that can be integrated into future navigation systems embedded into the c-arm, integrated within AR/MR headset, or in other commercial navigation systems.

The following sections provide, in Section 1, an overview of the significance of degrees of freedom, in Section 2, a definition of the ICE catheter and elaboration on the importance of roll angle prediction for its accurate tracking during cardiac catheterization. Additionally, the proposed machine learning-based model for roll angle prediction is described. In Section 3, the results of a study are presented, showcasing the accuracy of the disclosed model through various error criteria. In Section 4, certain key findings are summarized.

### Section 1: Overview

The term “6-DOF” refers to the six degrees of freedom of motion that a mechanism or virtual object is capable of exhibiting. These degrees of freedom include movement in the vertical (up-down) plane, the horizontal (left-right) plane, the longitudinal (forward-back) plane, as well as rotation about the x (roll), y (pitch), and z (yaw) axes. The ability to move in these six distinct ways is important for a variety of applications, such as in simulating the behavior of aircraft, robotics systems, and augmented/virtual reality (AR/VR) systems. The utilization of 6-DOF simulations is prevalent within the aerospace industry, serving as a valuable tool for both research and development, as well as for the training and evaluation of pilots and the assessment of aircraft designs. It is also crucial for robotic applications, as it enables the capability for the robotic arm to effectively access and manipulate objects in various positions and orientations. In industries such as manufacturing, transportation, and surgical procedures, the implementation of 6-DOF robotic arms allows for the successful completion of intricate tasks that would be difficult or impossible for humans to perform. The precise manipulation of objects is essential for tasks such as sorting, and 6-DOF robotic arms can be effective tools for such tasks.

In AR and VR as well as Mixed Reality (MR), 6-DOF allows for a greater level of realism as the user is able to move and interact with virtual objects in a similar way to how they would in the real world. For example, a 6-DOF controller allows the user to move their virtual hand in a natural, lifelike way, making it possible to grasp and manipulate virtual objects. AR and MR technologies can enhance minimally invasive surgery by providing improved visualization and precision during procedures. AR overlays virtual images and data onto the patient's body, while MR combines AR and VR to create an immersive experience for the surgeon. These technologies can also be used to train surgeons and provide remote assistance. They can improve patient outcomes by reducing the invasiveness of procedures and increasing the precision and skill of surgeons. Specially, the utilization of AR and MR technology in cardiac catheterization may be a valuable tool in improving diagnostic and therapeutic capabilities. The overlay of virtual images of the heart and its vasculature onto a real-time 3D representation of the patient's anatomy allows for enhanced visualization of the heart's structure, thereby facilitating the precise navigation and guidance of the catheter during the procedure. This can result in an increase in the safety and efficiency of the procedure. Furthermore, the use of AR and MR technology can aid in the planning and execution of complex procedures such as transcatheter aortic valve implantation (TAVI) and transcatheter mitral valve replacement (TMVR).

Although many cardiac catheters are tube-like objects that only require 5-DOF, there are many that are rotationally asymmetric and thus 6-DOF tracking can be critical. In order to collect the necessary data for 6-DOF tracking of catheters, two options are available. The first option involves the integration of two receiving coil probes of electromagnetic (EM) sensors into the tip of the catheter. This method offers portability, but has a low accuracy of up to ~5 mm and may result in out-of-field data. Additionally, the hardware required for this method is not readily available in catheterization labs, and manual integration of the probes into the catheter tip can introduce additional errors or the need for specialized equipment that is not scalable, given the FDA requirements for such procedures. Furthermore, this method is limited to specific types of catheters and is not a general solution for 6-DOF tracking.

The second option for 6-DOF tracking involves the use of real-time bi-plane fluoroscopy imaging. This method offers a general tool that can be used for all types of catheters and X-ray fluoroscopy machines are widely available in catheterization labs, albeit bi-plane c-arms are less common. However, this method only offers 5-DOF tracking and does not directly provide roll angle information, which is crucial for catheterization procedures. Roll angle is an essential parameter of 6-DOF tracking in catheterization, such as when using Intracardiac Echocardiography (ICE) catheters. It provides precise navigation of the catheter by rotating it to access different parts of the heart and avoid obstacles, better visualization of the heart and surrounding structures by changing the view direction of the ultrasound transducer, increased flexibility in catheter positioning, and reduction in procedure time by easily obtaining the best imaging view.

To rectify the lack of roll angle sensing capabilities and to acquire complete 6-DOF tracking data for AR/MR systems, various embodiments of the disclosed approach provide a machine learning-based model to predict roll angles (e.g., of an ICE catheter or other instrument) based on the positional tracking of the instrument (e.g., the ICE catheter or otherwise) using one or more medical imaging modalities (e.g., bi-plane fluoroscopy imaging). In example embodiments, a Multi-Input and Single Output (MISO) machine learning-based universal approximator can accurately predict the roll angle of a catheter using inputs derived from coordination of two intrinsic markers extracted from the synchronous frames of videos obtained from Antero Posterior (AP) and Left Anterior Oblique at 90 degrees (LAO90) planes of fluoroscopy imaging.

### Section 2: Materials and Methods

### 2.1. ICE Imaging and the Utility of Roll Angle Prediction

ICE is a real-time imaging modality that provides high-resolution visualizations of cardiac structures and allows for continuous monitoring of catheter positioning within the heart. It is well-tolerated by patients and has a reduced need for fluoroscopy and general anesthesia. It is commonly used for procedures, such as atrial septal defect closure and catheter ablation of cardiac arrhythmias and has an expanding role in other procedures. ICE imaging uses ultrasound technology to produce images of the inside of the heart. A small, flexible catheter with a transducer at the tip is inserted into a blood vessel and guided to the heart. The transducer emits high-frequency sound waves, which bounce off the heart structures and return to the transducer as echoes. These echoes are then converted into images that can be viewed on a monitor.

The roll angle is a crucial parameter that impacts the accuracy of the ICE imaging process during cardiac procedures. It influences the viewing orientation of the transducer that will affect the speed, safety, and outcome of the procedure. The field of view of an ICE catheter is 90°, and thus, a 5-15° degree error will not significantly impact the physician's ability to orient the catheter during the procedure, given they are still able to visualize the live display of the ICE monitor. However, if being used in a closed-loop robotic system, a wrong angle can lead to undesired consequences, such as a misdiagnosis, puncture, or improper delivery of a device. Similarly, the accurate prediction and display of the roll angle within an AR/MR system is imperative for providing the physician with the best possible representation of the catheter's position and orientation during a procedure. Moreover, this technology can also provide an interactive platform for physicians to collaborate and discuss the patient's condition and treatment plan, leading to improved patient outcomes.

### 2.2. Roll Angle Prediction

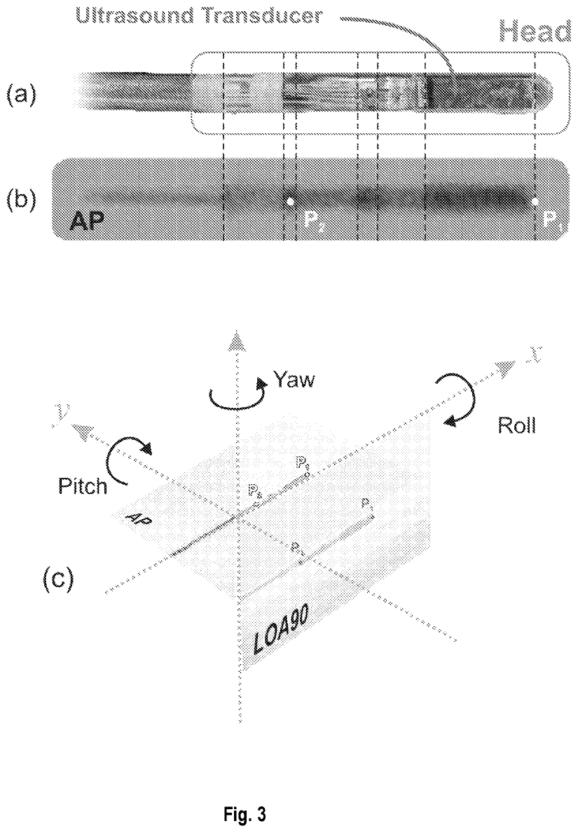

As illustrated in FIG. 3, at (a) and (b), the ICE catheter sensor head features three radiopaque markers which are clearly visible on fluoroscopy imaging ((b) in FIG. 3). Using the first marker in the AP (X-Y plane) and LAO90 (X-Z plane) planes ((c) in FIG. 3), the tip of catheter (P₁) can be tracked in both planes and the 3 positional degrees of freedom for the tip (X₁, Y₁, and Z₁) determined. Additionally, by calculating the angle of P₁ around center of one of the other markers (P₂ was chosen for this study) in the AP and LAO90 planes, the 2 angular degrees of freedom, Yaw (ψ) and Pitch (θ), respectively, can be determined. However, to determine the roll angle (φ), a third plane perpendicular to the x-axis (Y-Z plane) is required. Unfortunately, this plane is not visible on bi-plane fluoroscopy.

Through analysis of data obtained from bi-plane fluoroscopy, an empirical nonlinear correlation between the current 5-DOF data (X₁, Y₁, Z₁, ψ, and θ) and the remaining 6th degree of freedom (φ) was obtained. Various embodiments model this correlation in order to predict the value of φ. Based on the above-mentioned description, the relationship between q and the variables X₁, Y₁, Z₁, ψ, and θ can be expressed mathematically as follows:

$$φ = {f}_{1} ( {X}_{1} , {Y}_{1} , {Z}_{1} , ψ , θ ) \qquad ( 1 )$$

In order to calculate ψ and θ, the coordinates of P₁ and P₂ in both planes are utilized. ψ represents the angle of P₁ around P₂ on the AP plane, while θ represents the angle of P₁ around P₂ on the LAO90 plane. Mathematically, ψ is a function of X₁, X₂, Y₁, and Y₂, like g₁(X₁, X₂, Y₁, Y₂) and θ is a function of X₁, X₂, Z₁, and Z₂ like g₂(X₁, X₂, Z₁, Z₂). This results in the following equation:

$$φ = {f}_{1} ( {X}_{1} , {Y}_{1} , {Z}_{1} , {g}_{1} ( {X}_{1} , {X}_{2} , {Y}_{1} , {Y}_{2} ) , {g}_{2} ( {X}_{1} , {X}_{2} , {Z}_{1} , {Z}_{2} ) ) \qquad ( 2 )$$

where g₁ and g₂ are defined as follows:

$$ψ = {g}_{1} ( {X}_{1} , {X}_{2} , {Y}_{1} , {Y}_{2} ) = Atan 2 ( ( {Y}_{1} - {Y}_{2} ) , ( {X}_{1} - {X}_{2} ) ) \qquad ( 3 )$$

$$θ = {g}_{2} ( {X}_{1} , {X}_{2} , {Z}_{1} , {Z}_{2} ) = Atan 2 ( ( {Z}_{1} - {Z}_{2} ) , ( {X}_{1} - {X}_{2} ) ) \qquad ( 4 )$$

The A tan 2(a, b) function is an arctangent function that differs from the A tan(·) function in that it can handle all possible values of a and b, including negative values and zero. Unlike the A tan function, which is limited to the first and fourth quadrants, the A tan 2 function can determine the angle of a point in all four quadrants.

Based above definition, it can be inferred that φ is solely a function of the positional coordinates of P1 and P2 in both planes, and can be expressed as follows:

$$φ = {f}_{2} ( {X}_{1} , {Y}_{1} , {Z}_{1} , {X}_{2} , {Y}_{2} , {Z}_{2} ) \qquad ( 5 )$$

Where ƒ₂ is a nonlinear function that can be implemented by any universal approximator. In this study, we have employed a neural network-based universal approximator, which is detailed in the subsequent section.

### 2.3. Proposed ML-Based Model for Roll Angle Prediction

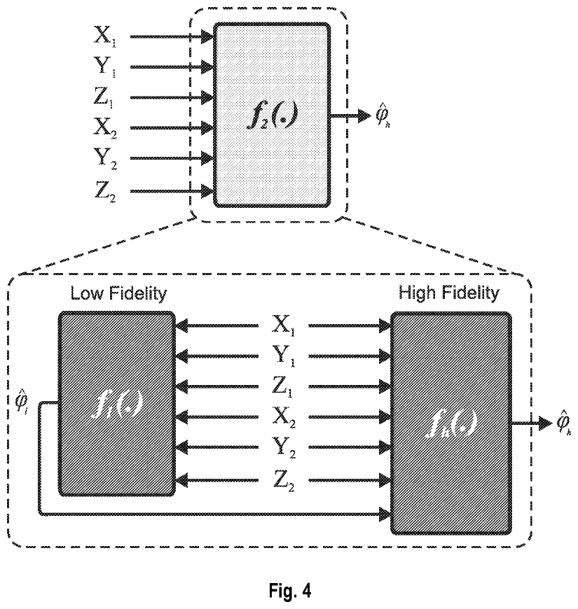

The objective of embodiments of the disclosed model is to identify the ƒ₂ functions in equation 3. A variety of approaches, including neural networks, fuzzy systems, neuro-fuzzy systems, nonparametric Volterra-based models, wavelet models, etc. can be employed to realize ƒ₂ in various embodiments. In example embodiments, a two-cascade deep fully connected neural network, as depicted in FIG. 4, may be employed for the prediction of roll angle in an ICE catheter. The arguments of the ƒ₂ functions serve as inputs for the disclosed model, with the output being represented by q. In example embodiments, the model comprises two compartments, a low-fidelity model (LF) and a high-fidelity model (HF). All six inputs are inputted into the LF model, which produces a low-fidelity prediction of φ (denoted as \hat{φ}ₗ). Subsequently, all six inputs and \hat{φ}ₗ are inputted into the HF model to produce a more accurate prediction of φ (denoted as \hat{φ}ₕ).

An example embodiment of the model consists of two fully connected deep neural networks (LF and HF) with five hidden layers, each containing ten neurons. In other embodiments, the model may include (but not be limited to) deep neural networks with a suitable number of hidden layers, each containing a suitable number of neurons. No dropout was applied to the networks during the training process in this example embodiment. The LF compartment was trained first to meet one of the early stopping criteria, which include a maximum of 1000 epochs, a minimum performance gradient of 1e-6, zero mean squared error, and six consecutive validation failures. After training the LF model, its output was used as the input to the HF compartment, which was then trained with the same early stopping criteria. This sequential training method may provide low error. In some embodiments, the training method may result in a lack of generalization for rare samples, resulting in a larger standard deviation in absolute error. To address this issue, both training procedures may be placed in a while loop, allowing the models to be trained multiple times with different initial random weights to achieve low error and low standard deviation. The Levenberg-Marquardt backpropagation training method may be used to train the LF and HF compartments using, for example, 70% of the shuffled dataset, and the models may be validated using the remaining 20% to minimize overfitting. Finally, the entire LF/HF model may be tested on the remaining 10% of the dataset that was not seen during the training process.

### 2.4. Input Feature Extraction

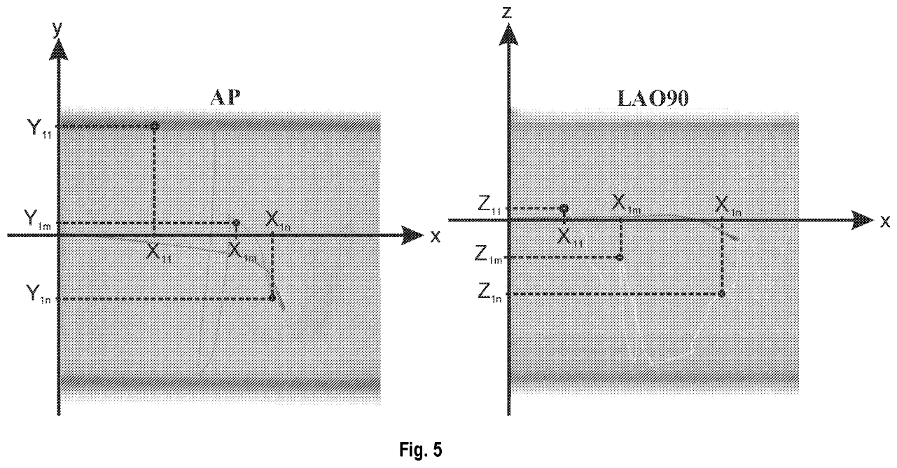

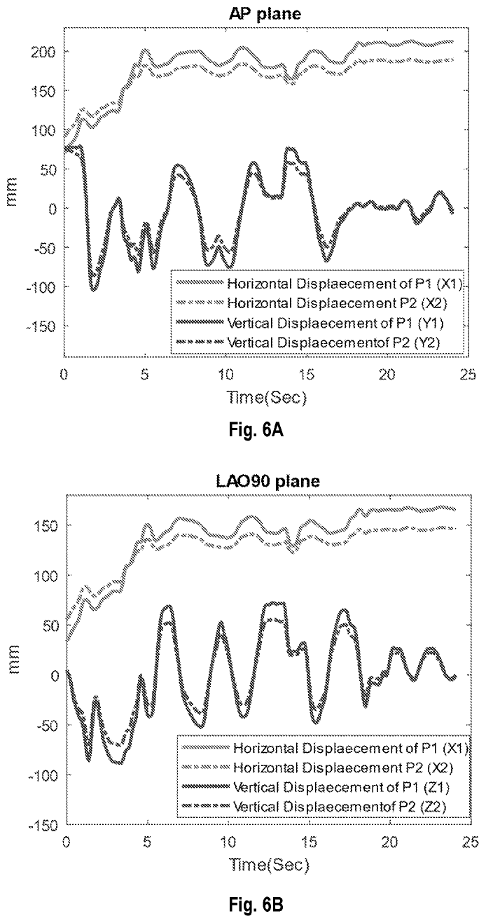

The ƒ₂ function, as specified in equation 3, employs six input features (X₁, Y₁, Z₁, X₂, Y₂, Z₂) derived from both the AP and LAO90 views of the saved fluoroscopy video. The procedure for extracting these features for three samples (the first, mth and nth frames) at point P₁ has been depicted in FIG. 5. The same process can be applied to point P2. As shown in FIG. 5, vectors for P₁ (X₁, Y₁, Z₁) and P₂ (X₂, Y₂, Z₂) are comprised of scalar values extracted from the video frames. These scalars can be obtained through custom image processing-based point tracking or utilizing open-source software. In an example case, Kinovea open-source software was utilized to automatically track P₁ and P₂, returning the horizontal and vertical displacement of both points over time. It should be noted that the horizontal displacement in both AP and LAO90 views correspond to the X axis in the 3D representation of FIG. 1, at (c), with one view omitted as they exhibit nearly 99% correlation. Additionally, vertical displacement in the AP plane represents the Y axis, while vertical displacement in the LAO90 plane represents the Z axis. The extracted input features have been shown in FIGS. 6A and 6B.

### 2.5. Output Feature Extraction

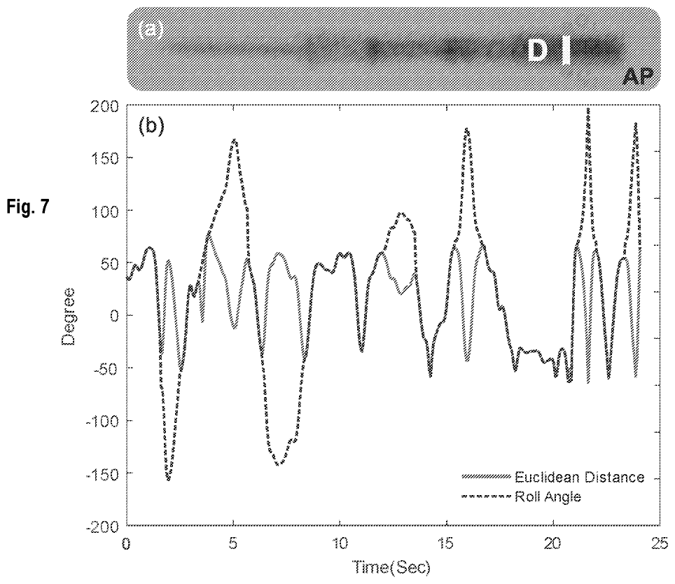

The output feature of the proposed LF/HF model is Roll Angle (φ), which needs to be predicted using the proposed model. To train the model, ground truth data for φ is required, for which the Human-in-the-loop Labeling (HITLL) method was utilized. The length of the black area of the ICE catheter tip on the AP view can be extracted by calculating the Euclidean distance (D) between two tracked points at the edges of this region (q₁, and q₂), as shown in FIG. 7 at (a). These points can be tracked using the Kinovea open-source software, but with manual effort. This step is performed only once to extract ground truth data and is not required later when using the trained model. A visual inspection of the video in the AP view reveals a direct relationship between D and φ. While D is highly correlated with the magnitude of φ, it does not contain information about its direction, which needs to be manually determined. By analyzing the D graph and observing the recorded video in the AP plane, it can be determined that in some frames, D is equal to φ, while in the rest of the frames, φ is equal to the vertically flipped version of D. As a result, as shown in FIG. 7 at (b), the human-in-the-loop feature extraction process involves vertically flipping the D graph in selected samples to obtain ground truth data for q.

### Section 3: Results

The disclosed example model was trained using 360 paired bi-plane fluoroscopic images obtained during mock procedures in a catheterization lab. These images depict the maneuvering of a VersiSight Pro ICE catheter (Philips) in both AP, and LAO90 planes. As was described previously, the data was partitioned into three subsets, with 70% (252 images) allocated as the training set, 20% (72 images) as the validation set, and 10% (36 images) as the testing set. The training and validation sets were utilized during the model training phase, while the testing set was reserved for evaluating the model's performance at the conclusion of the training phase. To ensure a representative distribution of the ICE catheter's movement in both the training and testing datasets and prevent overfitting, the data was randomly shuffled prior to being divided into the training and testing sets.

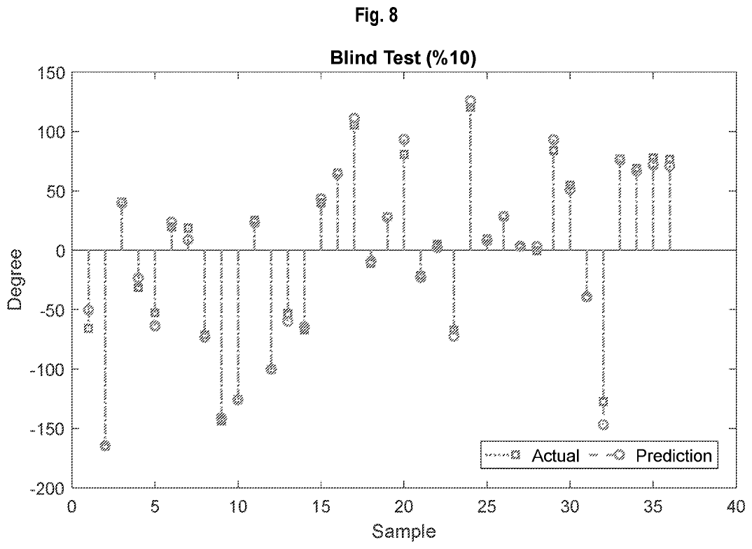

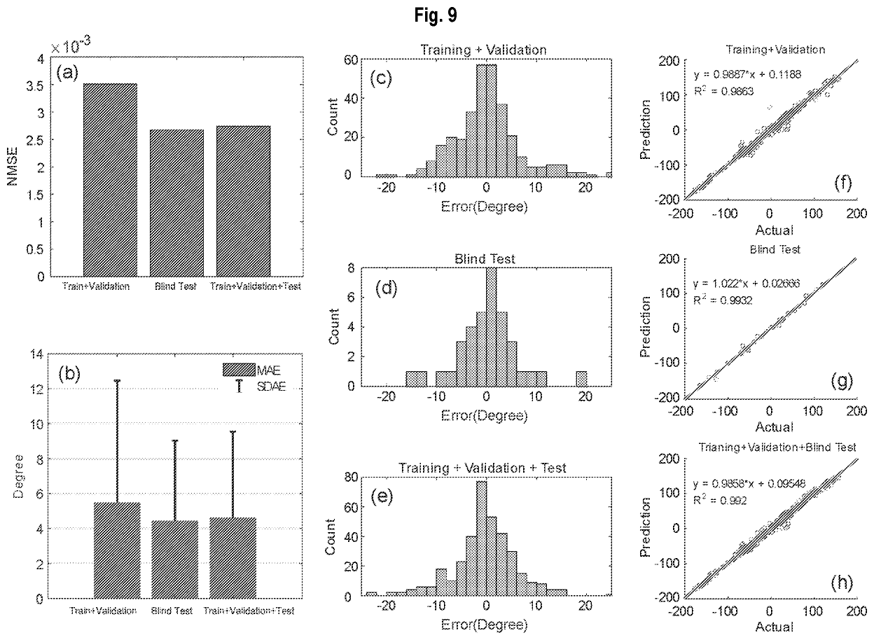

An example process of system identification includes three stages: data creation (feature extraction), model determination, and validation. The first two stages are described in Section 2. In this section, the focus is on the validation stage, where the model is tested on a portion of the dataset that it has not seen before. The results, as depicted in FIG. 8, indicate that the proposed model closely follows the output with high accuracy. To quantitatively assess the performance of the method, three metrics are selected: Normalized Mean Square Error (NMSE), Mean Absolute Error (MAE), and Standard Deviation of Absolute Error (SDAE). In FIG. 9, at (a) and (b), the values of NMSE, MAE, and SDAE are depicted for three sample groups: Training and Validation samples, Blind test samples, and all samples (Training, Validation, and Blind test). As displayed, the NMSE values are of the order of ~3×10⁻³, indicating a low error and a high level of fitting. The MAE values are of the order of ~4.5 degrees of error in the prediction of the roll angle, corresponding to ~1.25% error on average (~4.5/360 degree), which is considered acceptable for practical purposes. Finally, the SDAE values show a standard deviation of less than 5 degrees, implying that the model is capable of predicting the roll angle of the ICE catheter with a deviation of approximately 1.39% or 5/360 degree, which is also acceptable for surgeons. In FIG. 9, at (c), (d), and (e), the absolute error histograms of the model are displayed for the same three sample groups. All three histograms exhibit a normal distribution centered around zero, indicating that the majority of errors are less than 5 degrees, which is acceptable for catheterization procedures. To demonstrate the linear correlation between the predicted and actual outputs of the model, a line was fitted over the data for each of the three sample groups in FIG. 9, at (f), (g), and (h). The R-squared values for all three groups are approximately 0.99, indicating a high degree of accuracy in replicating the actual output by the proposed model.

### Section 4: Summary of Certain Example Embodiments

Catheterization is a procedure used to diagnose and treat various cardiovascular diseases. Intracardiac echocardiography (ICE) is an emerging imaging modality that has gained popularity in cardiac catheterization procedures due to its ability to provide high-resolution images of the heart and its surrounding structures in a minimally invasive manner. However, given its limited field of view, understanding its orientation within the heart is difficult simply from observing the acquired images. Therefore, ICE catheter tracking would be useful as guide during the procedure. There are two traditional tracking methods for 3D tracking, (1) Electro-Magnetic (EM) sensors and (2) Bi-plane fluoroscopy. EM sensors offer the convenience of portability and real-time tracking, but their accuracy is limited to approximately 5 mm and they are restricted to certain types of catheters. Hence, they may not be well-suited for all cardiac catheterization procedures. For the second option, we have bi-plane fluoroscopy, and although it is more accurate than EM sensors and compatible with all catheter types, it only provides 5-DOF tracking, thereby omitting information about the roll angle of the catheter.

To overcome these limitations, various embodiments can employ a machine learning-based approach to predict the roll angle of an ICE catheter using bi-plane fluoroscopy images to have a full 6 DOF tracking system. The innovative approach of using machine learning models to track the catheter has several advantages over traditional EM sensors. Machine learning algorithms can analyze vast amounts of data and identify complex patterns that may be difficult for traditional sensors to detect. Additionally, this approach does not require any additional hardware, making it a cost-effective solution for catheter tracking.

The results of the study of the example embodiments showed that the developed example model has a low error rate and a high degree of accuracy with Normalized Mean Square Error values of ~3×10⁻³, an average error of ~1.25% with Mean Absolute Error values of ~4.5 degrees, and a standard deviation of less than 5 degrees. Additionally, the model demonstrated a high degree of accuracy with R-squared values of approximately 0.99.

In other embodiments, the model may be trained on clinical images with a greater complexity of background features to improve its accuracy in real-world scenarios. Moreover, the model may be combined with co-registration algorithms that align the ICE catheter to the patient's anatomy for VR/AR/MR applications. Further, a larger dataset that extracts values from images acquired from various machines, sites, and settings can be used to validate the model's generalizability.

The results of the study of the example embodiments demonstrate that the developed model can provide 6-DOF tracking of an ICE catheter, enhancing the accuracy of VR/AR/MR-based real-time guidance systems that provide visualization and navigation during clinical procedures. This has significant implications for cardiac catheterization procedures, which are increasingly relying on these advanced imaging modalities. Accurate tracking of the medical instruments during procedures will not only improve the accuracy of diagnoses and treatments but also reduce the risks and complications associated with the procedures.

In other embodiments, feature extraction may be incorporated through image analysis on clinical data. Example embodiments of the predictive model employ scalar data obtained via open-source tracking software as its inputs, which does not necessitate feature extraction via image analysis. In other embodiments, to facilitate the operation of the current model, a deep learning-based model that automatically extracts the relevant features from clinical images may be employed. Multiple modules including the automatic feature extraction module, predictive model, and VR/AR/MR unit, may be integrated into a real time 6DOF tracking and visualization system (e.g., as depicted in FIG. 1).

The disclosed approach is further illustrated by the following example embodiments, which should not be construed as limiting in any way.

In various embodiments, the model's accuracy in predicting roll angles and may be enhanced, improving its overall robustness and generalizability, if additional data can be gathered and necessary features extracted. In distinct experiments, the ICE catheter was positioned in various regions of interest along the heart boundary. By concatenating all the acquired data, comprehensive coverage of the heart boundary was achieved, providing the model with a greater opportunity for training across various heart locations. This approach can enhance the model's robustness and generalizability. In some embodiments, a more complex model may be suitable due to the increased variability the model must predict.

### Section 5: Computer Vision-Based Catheter Segmentation and 3D Trajectory Extraction

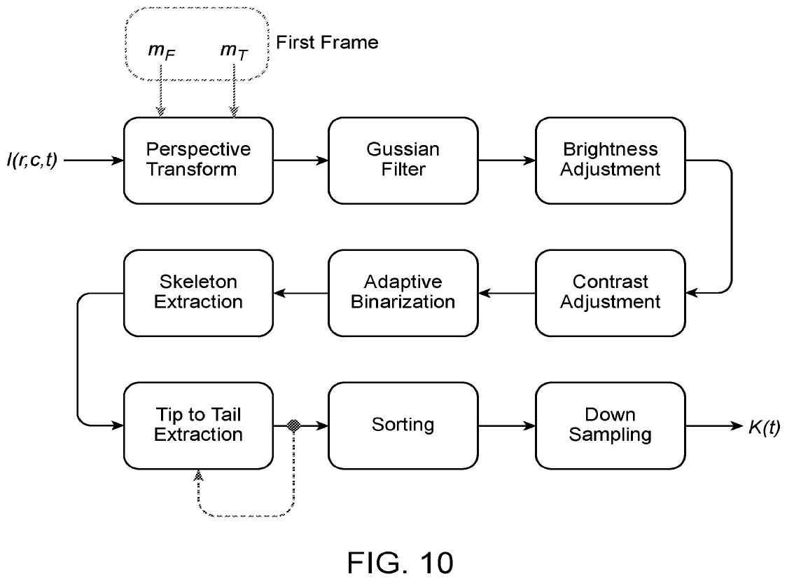

In example embodiments, a component enabling real-time tracking of the catheter position is a computer vision segmentation algorithm that first identifies and segments the catheter and then reconstructs the full 3D trajectory from synchronized orthogonal camera footage captured from the workspace. The processing pipeline operates on a per-frame basis, taking in video streams from two cameras positioned orthogonally around the 3D cube model. The cameras are intrinsically calibrated and have known, fixed poses relative to the prespecified coordinate space. An example vision algorithm with nine steps is illustrated in FIG. 10.

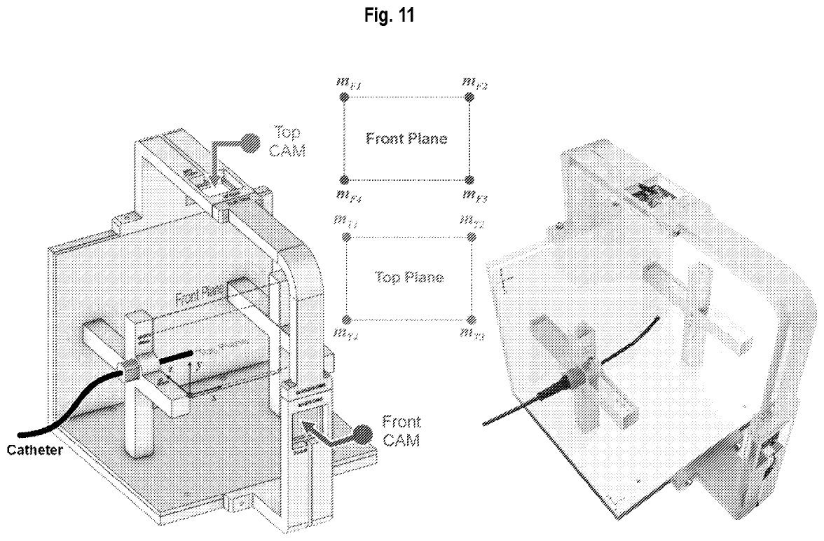

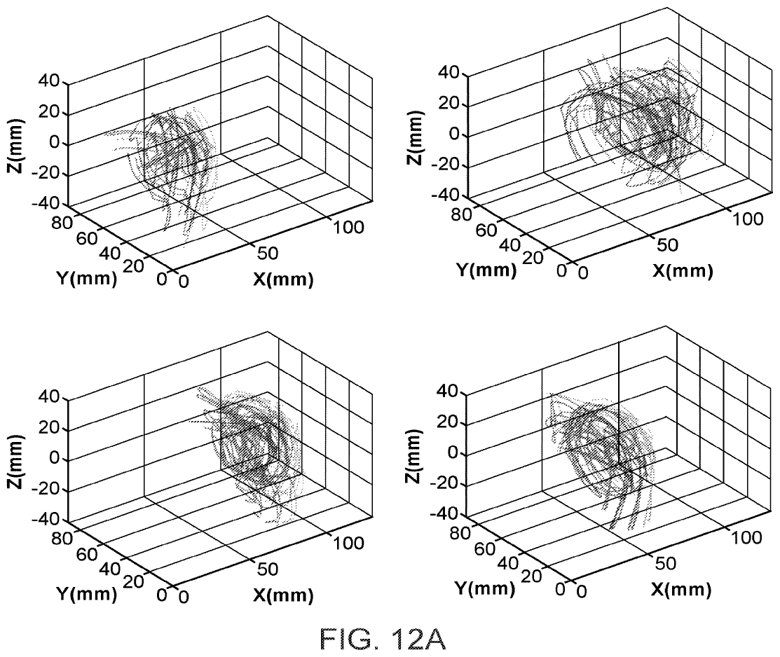

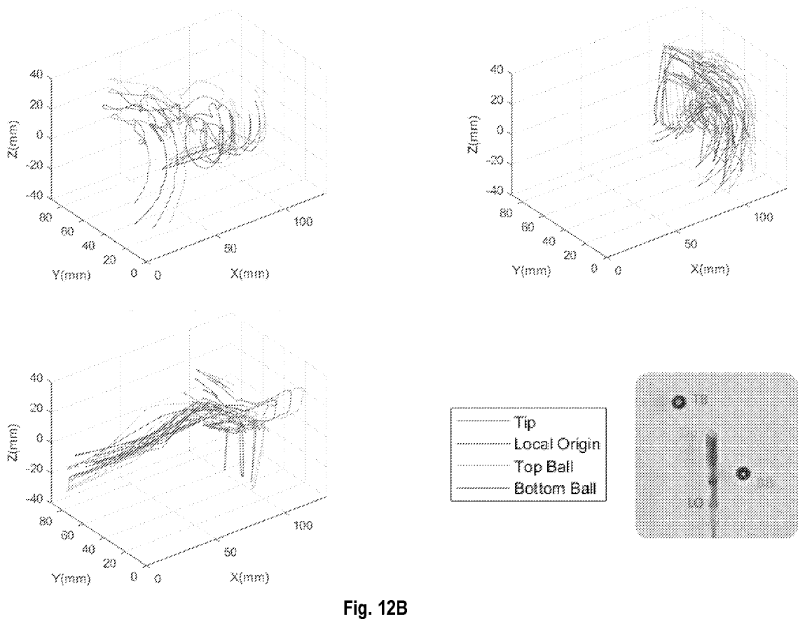

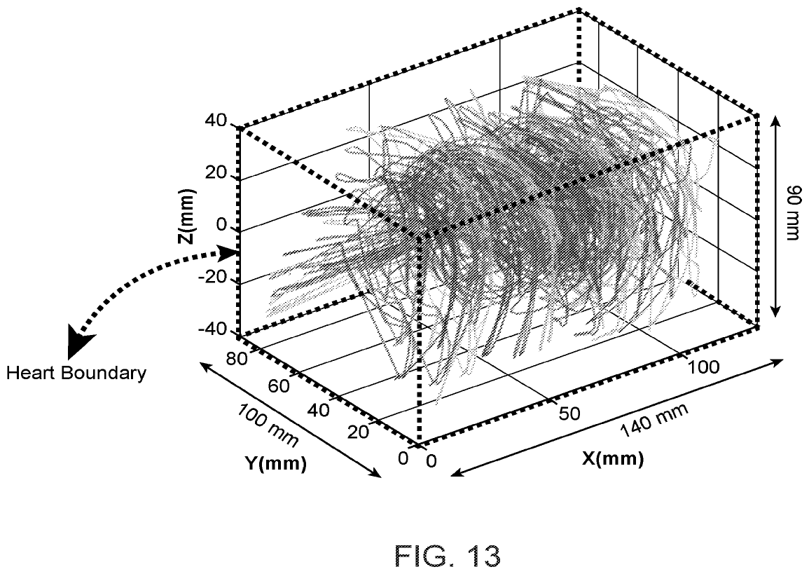

Each frame of the real-time video is denoted by I(r, c, t), where r, c, and t represent the pixel row, pixel column, and time dimensions, respectively. To start, every frame undergoes a perspective transformation using a perspective transformation matrix (Equation 6) mapping the front and the top cameras to the same global coordinate space, based on the known fiducial points (denoted by m_{T}, and m_{F}). Vectors m_{T} and m_{F}, each consisting of eight coordinate values (four pairs, m_{Ti} and m_{Fi} for i=1, 2, 3, and 4 as depicted in FIG. 11), may be used to specify the four corners of the region of interest (ROI) for the top and front planes, respectively. These vectors are marked on the two printed crosses within the setup (FIG. 11). The perspective transformation facilitates consistent image processing in the unified coordinate space and cancels out the distortion. As expressed in Equation 6, the transformed coordinate points (x′, y′) are obtained from the local camera-based coordinates (x, y). The coordinate transformation matrix implements an affine transformation using rotation and scaling (a1 to a4), translation (b1 and b2), and projection (c1 and c2). The parameters of the transformation matrix are initially unknown. To obtain these parameters, a set of eight equations need to be solved. To do so, as an initial step, four fiducial points are selected within the input image. Subsequently, these predefined points are mapped to predetermined locations based on the known dimensions and positions of ROIs. This procedure yields a system of eight equations and eight unknowns, allowing for a solvable configuration. The resulting perspective transformation matrix can then be computed. After the acquisition of the transformation matrix, all the input frames undergo perspective transformation, yielding coordinate-normalized images. Matrix parameters are acquired using the initial frame of each video, enabling seamless application across subsequent frames in real-time scenarios.

$$[ \begin{matrix}{X}^{′} \\ {Y}^{′} \\ 1\end{matrix} ] = [ \begin{matrix}{a}_{1} & {a}_{2} & {b}_{1} \\ {a}_{3} & {a}_{4} & {b}_{2} \\ {c}_{1} & {c}_{2} & 1\end{matrix} ] [ \begin{matrix}X \\ Y \\ 1\end{matrix} ] \qquad ( 6 )$$

In example embodiments, with the frames aligned, preprocessing steps are applied including Gaussian smoothing to reduce background noise and enable robust detection; a larger Gaussian kernel size increases the smoothness but can also degrade localization precision. Next, contrast/brightness adjustments enhance the visibility of the catheter against the white background. To isolate the catheter, an adaptive thresholding operation is applied, which converts the grayscale frame into a binary image based on dynamic local thresholding. The adaptive thresholding algorithm computes individualized threshold value, denoted by T(x, y), for each pixel (x, y), by averaging the pixel values present in a localized neighborhood window centered on the target pixel. In other words, Adaptive thresholding is as follows:

$$T ( x , y ) = mean ( neighborhood ) - C \qquad ( 7 )$$

Where mean(neighborhood) is the mean of the pixel values within the neighborhood proximity window. To further normalize the pixel intensity threshold, the constant C, is subsequently subtracted from this mean value. C may be obtained empirically. Each pixel's final value, represented by g(x, y), is then obtained by masking the input frames using the computed threshold as in:

$$g ( x , y ) = \{ \begin{matrix}1 , & P V ( x , y ) > T ( x , y ) \\ 0 , & P V ( x , y ) \leq T ( x , y )\end{matrix} \qquad ( 8 )$$

Where the variable PV(x, y) denotes the pixel value within the input image. Consequently, the process involves binary classification of each pixel, wherein the adaptation to a locally determined threshold takes precedence over a globally assigned value for the entire image. The configurability of this method is manifested through the adjustable parameters of the neighborhood block size and the constant C. While larger block sizes serve to mitigate the impact of noisy artifacts, it is imperative to acknowledge that they concurrently introduce a trade-off by diminishing the precision of localization.

In the subsequent stage of the proposed vision algorithm, a morphological operation for skeletonization is introduced to attenuate the binary representation of the catheter object, shaping it into a central, pixel-wide arc that characterizes the medial axis trajectory. This transformation yields a concise portrayal, streamlining the subsequent tracking process. Distinct skeletonization methods are available, such as Zhang's method and Lee's method. In this instance, Zhang's method has been implemented, which involves a series of sequential passes across the image, systematically eliminating pixels situated on the periphery of the object. This iterative process persists until further removal of pixels becomes unattainable.

In example embodiments, subsequent to the skeletonization of the binary catheter, pixel coordinates are further calculated. Initially, the tip location is determined by identifying the point with the maximum number of neighboring true values (white pixels). The coordinates of the inferred tip are then recorded as the first data point within an array, with the corresponding pixels being set to true values in the skeletonized binary catheter image. This procedure is repeated iteratively until all pixels transition to true values, thereby documenting the coordinates of the entire catheter trajectory from its tip to its entry point. This yields a two-dimensional directional array (vector) that succinctly encapsulates the spatial trajectory of the catheter for each frame—from its tip to its entry point. These pixel coordinates are subsequently mapped onto a real-world 3D coordinate system in millimeters, utilizing known phantom dimensions and camera intrinsic parameters. The coordinates are further down sampled to generate a subset of K points, where the initial point denotes the catheter's tip, the concluding point designates the entry point, and the intervening K-2 points are evenly distributed to delineate the catheter's curvature. The computer vision algorithm may be implemented, in example embodiments, in Python using the OpenCV library and operates in real-time on both planes. The resulting K points infer the X, Y coordinates from the top plane and Z coordinate from the front plane. Combining these points results in a real-time 3D K-point tracking system, providing a holistic representation of the catheter's spatial configuration and its curvature.

### Section 6: Monoplane Prediction

Example embodiments of the model operate based on the positional coordinates of extracted features from the ICE catheter in two planes (X and Y from the AP plane, and Z from the LAO90 plane). However, during real surgeries, interventional specialists may prefer to adhere to a single plane (AP plane). Various embodiments can extend the system to remain functional with monoplane scenarios, providing a significant achievement from a clinical perspective. Theoretically, achieving 3D tracking of the catheter and calculating the roll angle using only a single plane is deemed impossible. Nevertheless, predicting Z coordinates by utilizing X and Y information is feasible through machine learning. This implies that, by predicting Z, example embodiments can approximate the 3D trajectory and Roll angle of the catheter using the predicted Z.

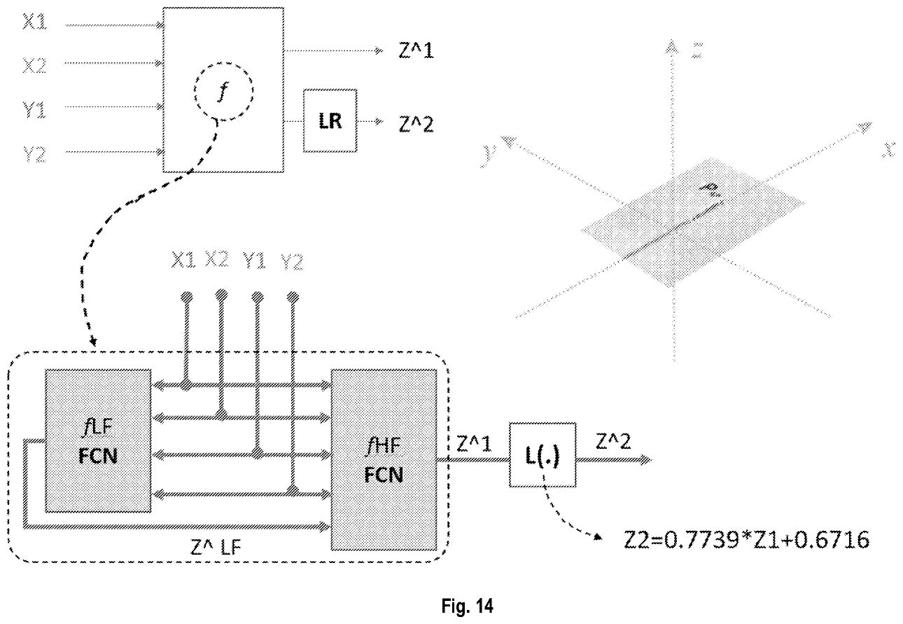

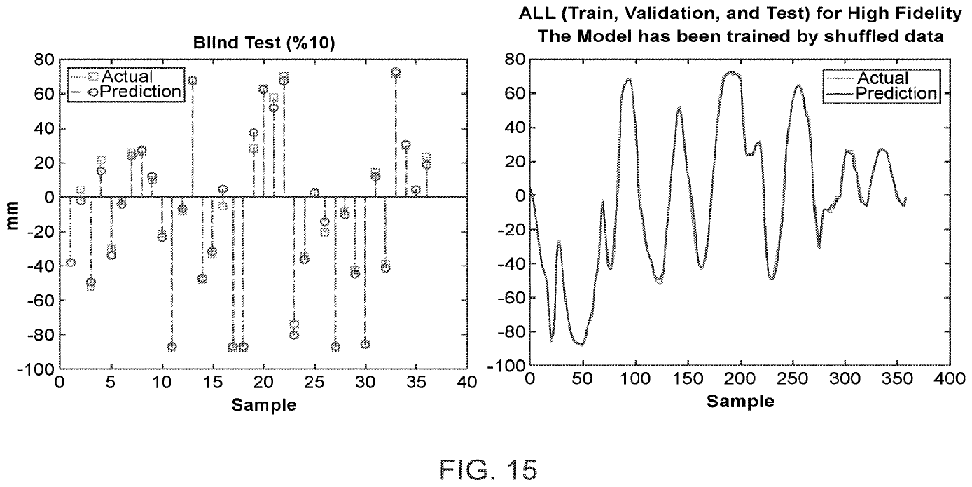

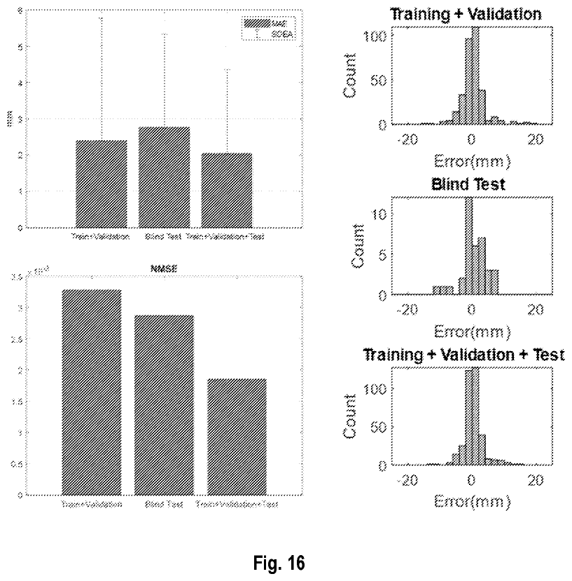

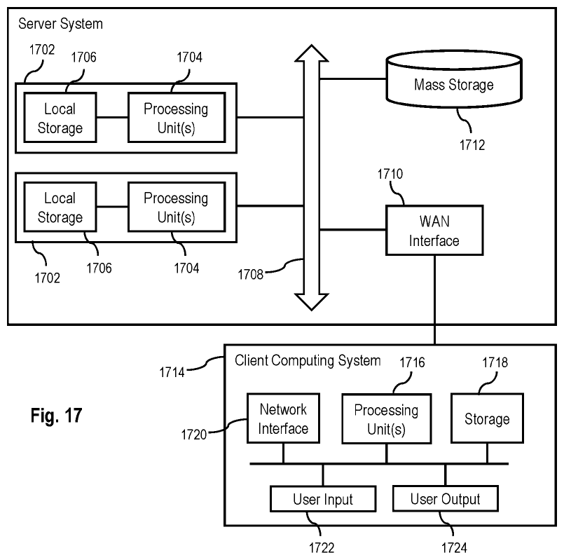

An example LFHF model for Z prediction (similar to models for roll prediction), as depicted in FIGS. 14-16. It can predict Z coordinates with an error of around 2 mm, which is deemed acceptable for 3D tracking but results in higher error when used as input for the roll angle prediction model. Hence, the current model can function for monoplane 3D tracking, but there is a need for increased accuracy in monoplane roll angle prediction

Various operations described herein can be implemented on computer systems having various configurations. FIG. 17 shows a simplified block diagram of a representative server system 1700 (e.g., computing system 110 or other components in FIG. 1) and client computer system 1714 (e.g., computing system 110, condition detection system 160, information system 170, and/or guidance system 180) usable to implement various embodiments of the present disclosure. In various embodiments, server system 1700 or similar systems can implement services or servers described herein or portions thereof. Client computer system 1714 or similar systems can implement clients described herein.

Server system 1700 can have a modular design that incorporates a number of modules 1702 (e.g., blades in a blade server embodiment); while two modules 1702 are shown, any number can be provided. Each module 1702 can include processing unit(s) 1704 and local storage 1706.

Processing unit(s) 1704 can include a single processor, which can have one or more cores, or multiple processors. In some embodiments, processing unit(s) 1704 can include a general-purpose primary processor as well as one or more special-purpose co-processors such as graphics processors, digital signal processors, or the like. In some embodiments, some or all processing units 1704 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 1704 can execute instructions stored in local storage 1706. Any type of processors in any combination can be included in processing unit(s) 1704.

Local storage 1706 can include volatile storage media (e.g., conventional DRAM, SRAM, SDRAM, or the like) and/or non-volatile storage media (e.g., magnetic or optical disk, flash memory, or the like). Storage media incorporated in local storage 1706 can be fixed, removable or upgradeable as desired. Local storage 1706 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 1704 need at runtime. The ROM can store static data and instructions that are needed by processing unit(s) 1704. The permanent storage device can be a non-volatile read-and-write memory device that can store instructions and data even when module 1702 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 1706 can store one or more software programs to be executed by processing unit(s) 1704, such as an operating system and/or programs implementing various server functions or any system or device described herein.

“Software” refers generally to sequences of instructions that, when executed by processing unit(s) 1704 cause server system 1700 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 1704. Software can be implemented as a single program or a collection of separate programs or program modules that interact as desired. From local storage 1706 (or non-local storage described below), processing unit(s) 1704 can retrieve program instructions to execute and data to process in order to execute various operations described above.

In some server systems 1700, multiple modules 1702 can be interconnected via a bus or other interconnect 1708, forming a local area network that supports communication between modules 1702 and other components of server system 1700. Interconnect 1708 can be implemented using various technologies including server racks, hubs, routers, etc.

A wide area network (WAN) interface 1710 can provide data communication capability between the local area network (interconnect 1708) and a larger network, such as the Internet. Conventional or other activities technologies can be used, including wired (e.g., Ethernet, IEEE 802.3 standards) and/or wireless technologies (e.g., Wi-Fi, IEEE 802.11 standards).

In some embodiments, local storage 1706 is intended to provide working memory for processing unit(s) 1704, providing fast access to programs and/or data to be processed while reducing traffic on interconnect 1708. Storage for larger quantities of data can be provided on the local area network by one or more mass storage subsystems 1712 that can be connected to interconnect 1708. Mass storage subsystem 1712 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 1712. In some embodiments, additional data storage resources may be accessible via WAN interface 1710 (potentially with increased latency).

Server system 1700 can operate in response to requests received via WAN interface 1710. For example, one of modules 1702 can implement a supervisory function and assign discrete tasks to other modules 1702 in response to received requests. Conventional work allocation techniques can be used. As requests are processed, results can be returned to the requester via WAN interface 1710. Such operation can generally be automated. Further, in some embodiments, WAN interface 1710 can connect multiple server systems 1700 to each other, providing scalable systems capable of managing high volumes of activity. Conventional or other techniques for managing server systems and server farms (collections of server systems that cooperate) can be used, including dynamic resource allocation and reallocation.

Server system 1700 can interact with various user-owned or user-operated devices via a wide-area network such as the Internet. An example of a user-operated device is shown in FIG. 17 as client computing system 1714. Client computing system 1714 can be implemented, for example, as a consumer device such as a smartphone, other mobile phone, tablet computer, wearable computing device (e.g., smart watch, eyeglasses), desktop computer, laptop computer, and so on.

Client computing system 1714 can communicate via WAN interface 1710. Client computing system 1714 can include conventional computer components such as processing unit(s) 1716, storage device 1718, network interface 1720, user input device 1722, and user output device 1724. Client computing system 1714 can be a computing device implemented in a variety of form factors, such as a desktop computer, laptop computer, tablet computer, smartphone, other mobile computing device, wearable computing device, or the like.

Processor 1716 and storage device 1718 can be similar to processing unit(s) 1704 and local storage 1706 described above. Suitable devices can be selected based on the demands to be placed on client computing system 1714; for example, client computing system 1714 can be implemented as a “thin” client with limited processing capability or as a high-powered computing device. Client computing system 1714 can be provisioned with program code executable by processing unit(s) 1716 to enable various interactions with server system 1700 of a message management service such as accessing messages, performing actions on messages, and other interactions described above. Some client computing systems 1714 can also interact with a messaging service independently of the message management service.

Network interface 1720 can provide a connection to a wide area network (e.g., the Internet) to which WAN interface 1710 of server system 1700 is also connected. In various embodiments, network interface 1720 can include a wired interface (e.g., Ethernet) and/or a wireless interface implementing various RF data communication standards such as Wi-Fi, Bluetooth, or cellular data network standards (e.g., 3G, 4G, 5G, LTE, etc.).

User input device 1722 can include any device (or devices) via which a user can provide signals to client computing system 1714; client computing system 1714 can interpret the signals as indicative of particular user requests or information. In various embodiments, user input device 1722 can include any or all of a keyboard, touch pad, touch screen, mouse or other pointing device, scroll wheel, click wheel, dial, button, switch, keypad, microphone, and so on.

User output device 1724 can include any device via which client computing system 1714 can provide information to a user. For example, user output device 1724 can include a display to display images generated by or delivered to client computing system 1714. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 1724 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 1704 and 1716 can provide various functionality for server system 1700 and client computing system 1714, including any of the functionality described herein as being performed by a server or client, or other functionality associated with message management services.

It will be appreciated that server system 1700 and client computing system 1714 are illustrative and that variations and modifications are possible. Computer systems used in connection with embodiments of the present disclosure can have other capabilities not specifically described here. Further, while server system 1700 and client computing system 1714 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. For instance, although specific examples of rules (including triggering conditions and/or resulting actions) and processes for generating suggested rules are described, other rules and processes can be implemented. Embodiments of the disclosure can be realized using a variety of computer systems and communication technologies including but not limited to specific examples described herein.

Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims. In some examples, these terms allow for a plus-or-minus deviation of 10 percent.

It should be noted that the terms “exemplary,” “example,” “potential,” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the Figures. It should be noted that the orientation of various elements may differ according to other exemplary embodiments, and that such variations are intended to be encompassed by the present disclosure.

The embodiments described herein have been described with reference to drawings. The drawings illustrate certain details of specific embodiments that implement the systems, methods and programs described herein. However, describing the embodiments with drawings should not be construed as imposing on the disclosure any limitations that may be present in the drawings.

It is important to note that the construction and arrangement of the devices, assemblies, and steps as shown in the various exemplary embodiments is illustrative only. Additionally, any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principles of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the terms “approximately,” “about,” “substantially,” and similar terms in reference to a number or value is generally taken to include numbers or values that fall within a range of 1%, 5%, or 10% in either direction (greater than or less than) of the number or value unless otherwise stated or otherwise evident from the context (except where such number would be less than 0% or exceed 100% of a possible value).

As used herein, the terms “individual”, “patient”, or “subject” are used interchangeably and refer to an individual organism, a vertebrate, a mammal, or a human. In a preferred embodiment, the individual, patient or subject is a human.

### EXAMPLES

Some sample embodiments are disclosed below, in order to represent illustrative embodiments, which one skilled in the art will understand may be further modified, combined, constrained, etc. according to the entirety of this disclosure.

Embodiment A1: A medical imaging system comprising one or more processors, the system configured to: receive, in real time, one or more medical images from an imaging device, the one or more medical images including one or more depictions of a medical instrument; determine, based at least in part on at least one of the received medical images, a roll angle of the medical instrument, the roll angle being indicative of a position of a reference feature of the medical instrument relative to a target; and generate a visualization based at least in part on the determined roll angle for presentation via a display device.

Embodiment A2: The medical imaging system of Embodiment A1, wherein the system is configured to determine the roll angle of the medical instrument based at least in part on a machine learning model.

Embodiment A3: The medical imaging system of Embodiment A2, wherein the machine learning model was trained based at least in part on groundtruth roll measurements.

Embodiment A4: The medical imaging system of Embodiment A3, wherein the groundtruth roll measurements were acquired from pairs of images taken at two different angles.

Embodiment A5: The medical imaging system of either Embodiment A3 or A4, wherein the groundtruth roll measurements were acquired using electromagnetic sensors.

Embodiment A6: The medical imaging system of any of Embodiments A3-A5, wherein the groundtruth roll measurements were acquired using impedance sensors.

Embodiment A7: The medical imaging system of any of Embodiments A3-A6, wherein the groundtruth roll measurements were acquired using ultrasound crystals.

Embodiment A8: The medical imaging system of any of Embodiments A3-A7, wherein the groundtruth roll measurements were acquired using fiber optics.

Embodiment A9: The medical imaging system of Embodiment A3, wherein the groundtruth roll measurements were acquired from one or more of, or any combination of: pairs of images taken at two different angles, electromagnetic sensors, impedance sensors, ultrasound crystals, and/or fiber optics.

Embodiment A10: The medical imaging system of any of Embodiments A2-A9, wherein the machine learning model was trained using a set of training data, each data point associated with a label indicative of roll angle for the medical instrument depicted in the image.

Embodiment A11: The medical imaging system of any of Embodiments A2-A10, wherein the machine learning model comprises one or more deep neural networks.

Embodiment A12: The medical imaging system of any of Embodiments A2-A11, wherein the machine learning model was trained to determine roll angle based on single images.

Embodiment A13: The medical imaging system of any of Embodiments A1-A12, wherein the roll angle is a first degree of freedom (DOF), the medical imaging system further configured to determine one or more additional degrees of freedom (DOFs).

Embodiment A14: The medical imaging system of any of Embodiments A1-A13, wherein the roll angle is a first degree of freedom (DOF), the medical imaging system further configured to determine a plurality of additional degrees of freedom (DOFs).

Embodiment A15: The medical imaging system of any of Embodiments A1-A14, wherein the roll angle is a first degree of freedom (DOF), the medical imaging system further configured to determine five additional degrees of freedom (DOFs).

Embodiment A16: The medical imaging system of any of Embodiments A1-A15, wherein the medical instrument is or comprises a minimally invasive tool.

Embodiment A17: The medical imaging system of any of Embodiments A1-A16, wherein the medical instrument is or comprises a catheter.

Embodiment A18: The medical imaging system of any of Embodiments A1-A17, wherein the medical instrument is or comprises imaging hardware.

Embodiment A19: The medical imaging system of any of Embodiments A1-A18, wherein the imaging hardware is or comprises endoscopic imaging hardware.

Embodiment A20: The medical imaging system of any of Embodiments A1-A19, wherein the one or more medical images are based at least in part on fluoroscopy.

Embodiment A21: The medical imaging system of any of Embodiments A1-A20, wherein the one or more medical images are based at least in part on ultrasound.

Embodiment A22: The medical imaging system of any of Embodiments A1-A21, wherein the one or more medical images are based at least in part on digital images captured using image sensors.

Embodiment A23: The medical imaging system of any of Embodiments A1-A22, wherein the one or more medical images are based at least in part on one or more of, or any combination of, fluoroscopy, ultrasound, and/or digital images captured using image sensors.

Embodiment A24: The medical imaging system of any of Embodiments A1-A23, wherein the visualization is for an augmented reality (AR) system that interfaces with the display screen, a second display screen, and/or with a headset.

Embodiment A25: The medical imaging system of any of Embodiments A1-A23, wherein the visualization is for a mixed reality (MR) system that interfaces with the display screen, a second display screen, and/or with a headset.

Embodiment A26: The medical imaging system of any of Embodiments A1-A23, wherein the visualization is for a virtual reality (VR) system that interfaces with the display screen, a second display screen, and/or a headset.

Embodiment A27: The medical imaging system of any of Embodiments A1-A23, wherein the visualization is for an extended reality (XR) system that interfaces with the display screen, a second display screen, and/or a headset.

Embodiment A28: The medical imaging system of any of Embodiments A1-A23, wherein the visualization is for a spatial computing system that interfaces with the display screen, a second display screen, and/or a headset.

Embodiment A29: The medical imaging system of any of Embodiments A1-A28, wherein the visualization is for one or more of, or any combination of, an augmented reality (AR) system, a mixed reality (MR) system, a virtual reality (VR) system, an extended reality system, and/or a spatial computing system that interfaces with the display screen, a second display screen, and/or a headset.

Embodiment B1: A method comprising: applying, in real time, a machine learning model to one or more medical images to determine a roll angle of a medical instrument depicted in the medical images; wherein the roll angle of the medical instrument is indicative of a position of a reference feature of the medical instrument relative to a target; and wherein the one or more medical images are based on one or more of fluoroscopy, ultrasound, or digital imagery.

Embodiment B2: The method of Embodiment B1, further comprising generating a visualization based at least in part on the determined roll angle for presentation via one or more of, or any combination of, a display device and/or a headset of an augmented reality (AR) system, a mixed reality (MR) system, a virtual reality (VR) system, an extended reality (XR) system, and/or a spatial computing system.

Embodiment B3: The method of either Embodiment B1 or B2, further comprising training the machine learning model.

Embodiment C1: A method comprising training a machine learning model to determine roll angle of a medical instrument based on single medical images.

Embodiment C2: The method of Embodiment C1, further comprising using the trained machine learning model to determine roll angle of the medical instrument.

Embodiment C3: The method of either Embodiment C1 or C2, wherein the machine learning model is used to determine roll angle of the medical instrument in real time or near real time during a medical procedure.

Embodiment D1: A method performed by a system and/or a device of any of Embodiments A1-A29.

Embodiment E1: A system and/or a device configured to perform any method of Embodiments B1-B3 or C1.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A medical imaging system comprising one or more processors, the system configured to:
receive, in real time, one or more medical images from an imaging device, the one or more medical images including one or more depictions of a medical instrument;
determine, based at least in part on at least one of the received medical images, a roll angle of the medical instrument, the roll angle being indicative of a position of a reference feature of the medical instrument relative to a target; and
generate a visualization based at least in part on the determined roll angle for presentation via a display device.

2. The medical imaging system of claim 1, wherein the system is configured to determine the roll angle of the medical instrument based at least in part on a machine learning model.

3. The medical imaging system of claim 2, wherein the machine learning model was trained based on groundtruth roll angle measurements, acquired from pairs of images taken at two different angles, electromagnetic sensors, impedance sensors, ultrasound crystals, or fiber optics.

4. The medical imaging system of claim 2, wherein the machine learning model was trained based using a set of training data, each data point associated with a label indicative of roll angle for the medical instrument depicted in the image.

5. The medical imaging system of claim 2, wherein the machine learning model comprises one or more deep neural networks.

6. The medical imaging system of claim 2, wherein the machine learning model was trained to determine roll angle based on single images.

7. The medical imaging system of claim 1, wherein the roll angle is a first degree of freedom (DOF), the medical imaging system further configured to determine one or more additional degrees of freedom (DOFs).

8. The medical imaging system of claim 1, wherein the roll angle is a first degree of freedom (DOF), the medical imaging system further configured to determine five additional degrees of freedom (DOFs).

9. The medical imaging system of claim 1, wherein the medical instrument comprises a minimally invasive tool, a catheter, or imaging hardware.

10. The medical imaging system of claim 1, wherein the one or more medical images are based on fluoroscopy.

11. The medical imaging system of claim 1, wherein the one or more medical images are based on ultrasound.

12. The medical imaging system of claim 1, wherein the one or more medical images are based on digital images captured using image sensors.

13. The medical imaging system of claim 1, wherein the visualization is for an augmented reality (AR) system, a mixed reality (MR) system, a virtual reality (VR) system, an extended reality (XR), or a spatial computing system that interfaces with the display screen, a second display screen, or a headset.

14. A method comprising:
applying, in real time, a machine learning model to one or more medical images to determine a roll angle of a medical instrument depicted in the medical images;
wherein the roll angle of the medical instrument is indicative of a position of a reference feature of the medical instrument relative to a target; and
wherein the one or more medical images are based on one or more of fluoroscopy, ultrasound, or digital imagery.

15. The method of claim 13, further comprising generating a visualization based at least in part on the determined roll angle for presentation via a display device of an augmented reality (AR) system, a mixed reality (MR) system, a virtual reality (VR) system, an extended reality (XR) system, or a spatial computing system.
